(19) United States
(12) Patent Application Publication
(10) Pub. No.: US 2012/0253164 A1
(43) Pub. Date: Oct. 4, 2012

(54) ELECTRODE SYSTEM FOR CRANIAL NERVE STIMULATION

(75) Inventors: Se-Ik Park, Pyeongtaek-si (KR); Yong Sup Hwang, Gyeongtaek-si (KR); Ho Yun, Pyeongtaek-si (KR); Jin-Woo Chang, Pyeongtaek-si (KR)

(73) Assignee: M.I.Tech Co., Ltd., Gyeonggi-do (KR)

(21) Appl. No.: 13/516,377

(22) PCT Filed: Apr. 28, 2010
(86) PCT No.: PCT/KR2010/002700

§ 371 Date: Jun. 15, 2012

(30) Foreign Application Priority Data
Dec. 15, 2009 (KR) 10-2009-0124540
Apr. 20, 2010 (KR) 10-2010-0036442

(51) Int. Cl. A61B 5/0478

(52) U.S. Cl. 600/383

(57) ABSTRACT

An electrode system for cranial nerve stimulation includes a support coupling part for coupling a support. The support coupling part has a stimulating electrode insertion part formed to couple a stimulating electrode at the bottom thereof. The electrode system further includes an electrode holder formed to couple to a connecting wire for supplying electricity to the stimulating electrode, and an anchor having an electrode holder insertion part formed with a hollow part into which the electrode holder coupled to the connecting wire is inserted, and fixed to the skull of a subject.

### FIELD OF THE INVENTION

The present invention relates to an electrode system for cranial nerve stimulation, and more particularly, to an electrode system for cranial nerve stimulation, which stably fixes a stimulating electrode of a deep brain stimulator to a subject such that the subject undergoes reliably a behavior experiment.

### BACKGROUND OF THE INVENTION

In order to develop medicines for pain treatment and movement disorders caused by diseases such as Parkinson's disease, pain, and epilepsy, a subject which is artificially made to have such diseases is used to perform a behavior experiment. The behavior experiment is to examine how much the state of the subject is improved while stimulating specific cranial nerves of the subject. However, when deep brain stimulation is performed on the subject, brain cells should not be destroyed by the electrical stimulation, and an electrode material and related devices should be safe enough to apply to organisms.

Meanwhile, laboratory white mice are typically used as the subject. In order to stimulate cranial nerves while satisfying the safety standard, an operation as illustrated in FIG. 1 may be performed. First, a skull 1 of a white mouse is incised, a metal electrode 6 formed of tungsten and coated with parylene at 5 μm is inserted into a target site 3 of a brain using an anchor 2 formed of stainless steel suitable for organisms, and a small amount of dental cement 4 is buried to stably fix the metal electrode 6. Then, one end of a connecting wire formed of a platinum-iridium alloy and molded of silicon elastomer is compressed and connected to the metal electrode, and the other end of the connecting wire is positioned under the skin of the subject and then exposed to the outside through the back of the subject. A socket is disposed at the end of the exposed portion so as to be coupled to a deep brain stimulator. After the connecting wire is positioned, the head of the white mouse, into which the anchor and the electrode are transplanted, is sutured using a suture fiber to reduce a risk of infection. The stimulator is mounted on the back of the white mouse by a fixing device 10 as illustrated in FIG. 2. The fixing device 10 includes a tether band 14 formed of an elastic body which is soft enough for the white mouse not to feel a pressure, and covers the top of a saddle 12 having the stimulator positioned thereon, thereby stably fixing the stimulator when the subject undergoes a behavior experiment.

In the deep brain stimulator of the prior art, however, when the subject moves its head or scratches the incised portion with its front paws during the experiment, a joint between the electrode and the connecting wire is frequently disconnected.

Furthermore, when the subject moves here and there inside a breeding house and bumps against the breeding house during the experiment, the deep brain simulator may be damaged. In this case, since it is impossible to transfer a desired stimulation signal to the subject, there are difficulties in deducing an experimental result.

Furthermore, when the subject is managed in the breeding house, the socket connected to the stimulator may be caught in the breeding house and then disconnected. When the above-described electrical disconnection occurs, the experiment is impossible to perform. Furthermore, since the behavior experiment requires a considerably long time, the efficiency of the behavior experiment decreases in terms of time and economy. Furthermore, the anchor formed of a metallic material may distort an image in an experiment using PET (Position Emission Tomography) or MRI (Magnetic Resonance Imaging) after the behavior experiment.

### SUMMARY OF THE INVENTION

It is, therefore, an object of the present invention to provide an electrode system for cranial nerve stimulation, which stably fixes a stimulating electrode with a simple structure such that an experiment may be performed reliably.

In accordance with an aspect of the present invention, there is provided an electrode system for cranial nerve stimulation, which includes: a support coupling part for coupling a support, wherein the support coupling part having a stimulating electrode insertion part formed to couple a stimulating electrode at the bottom thereat an electrode holder formed to couple to a connecting wire for supplying electricity to the stimulating electrode; and an anchor having an electrode holder insertion part formed with a hollow part into which the electrode holder coupled to the connecting wire is inserted, and fixed to the skull of a subject.

In accordance with another aspect of the present invention, there is provided an electrode system for cranial nerve stimulation, which includes: an anchor member configured to be fixed to the skull of a subject, wherein the anchor member includes a flange part having screw holes formed therein and an electrode holder formed integrally with the flange part such that a stimulating electrode is inserted into the electrode holder and supported by the electrode holder; and a stimulating electrode support screw coupled to the electrode holder of the anchor member so as to support the inserted stimulating electrode, and coupled to a connecting wire for supplying electricity.

In accordance with the embodiments of the present invention, the electrode system for cranial nerve stimulation has a simple structure, and stably fixes the stimulating electrode during an experiment even though a subject freely moves, thereby reliably performing the behavior experiment.

### BRIEF DESCRIPTION OF THE DRAWINGS

FIG. 1 schematically illustrates a deep brain stimulator of the prior art;

FIG. 2 is a perspective view of a fixing device for mounting the deep brain stimulator shown in FIG. 1 on a subject;

FIG. 3 is an exploded perspective view of an electrode system for cranial nerve stimulation in accordance with a first embodiment of the present invention;

FIG. 4 is a bottom view of the electrode holder shown in FIG. 3;

FIG. 5 is a cross-sectional view taken along line V-V of FIG. 3;

FIG. 6 is a perspective view illustrating the inside of a cover having protrusions to be coupled to an attachment groove formed on the outer circumference of an anchor;

FIG. 7 illustrates a state in which the electrode system is covered by a cover and buried in the skull of a subject;

FIG. 8 is a perspective view of a three-dimensional stereotaxic instrument for transplanting the electrode system shown in FIG. 3;

FIG. 9 is a perspective view of the support shown in FIG. 8;

FIG. 10 is an exploded perspective view of an electrode system for cranial nerve stimulation in accordance with a second embodiment of the present invention;

FIG. 11 is an assembled cross-sectional view of FIG. 10;

FIG. 12 is an exploded cross-sectional view of FIG. 10;

FIG. 13 illustrates a connection state of a connecting wire coupled to a stimulating electrode support screw shown in FIG. 10;

FIG. 14 is a perspective view of an electrode system for cranial nerve stimulation in accordance with a third embodiment of the present invention; and

FIG. 15 is an assembled plan view of FIG. 14.

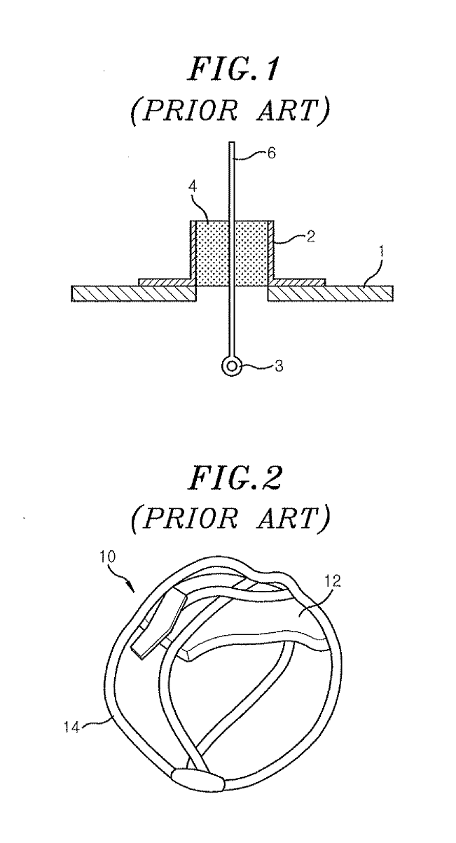

### DETAILED DESCRIPTION OF THE EMBODIMENTS

Hereinafter, embodiments of the present invention will be described in detail with reference to the accompanying drawings.

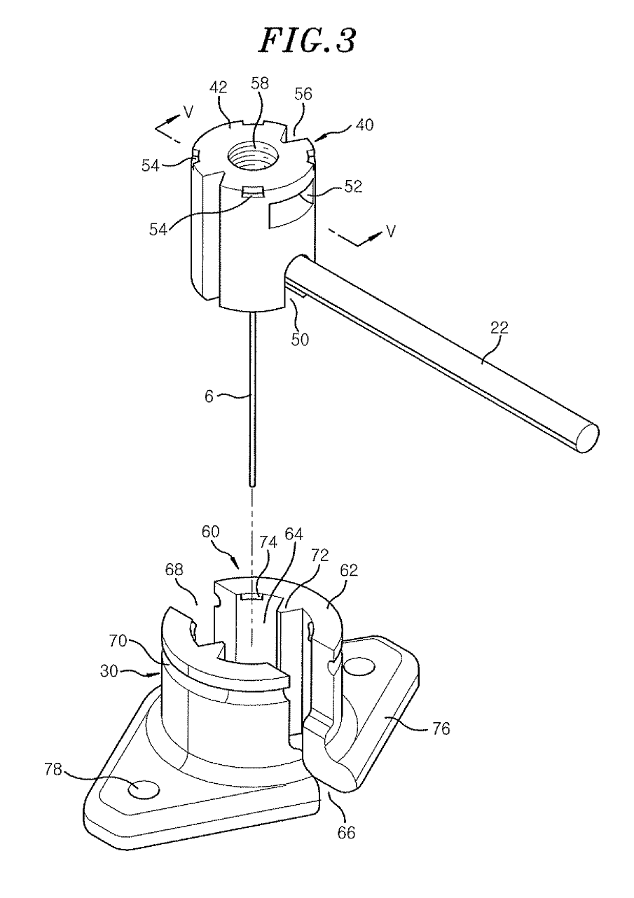

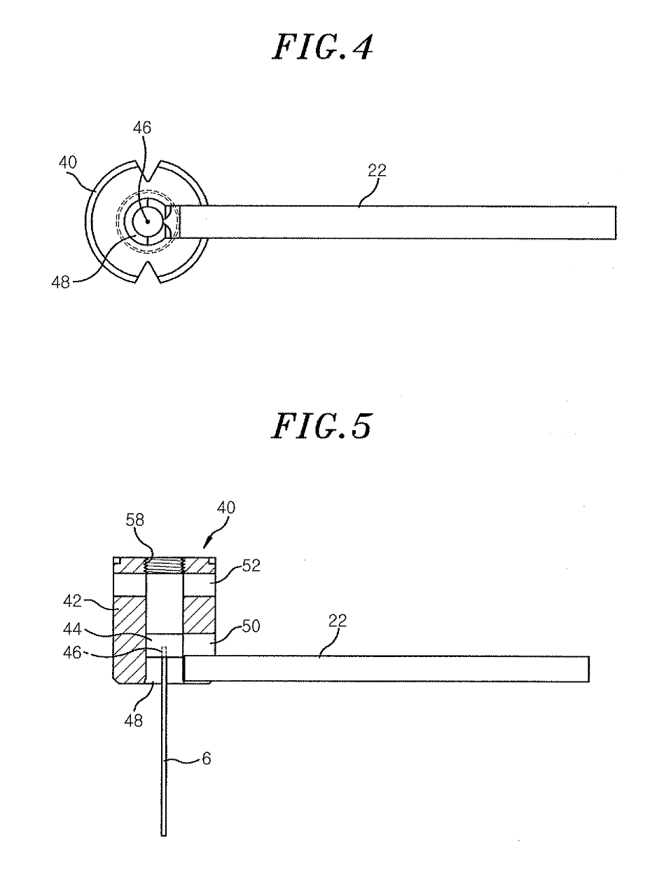

FIG. 3 is an exploded perspective view of an electrode system for cranial nerve stimulation in accordance with a first embodiment of the present invention, FIG. 4 is a bottom view of the electrode holder shown in FIG. 3, and FIG. 5 is a cross-sectional view taken along line V-V of FIG. 3.

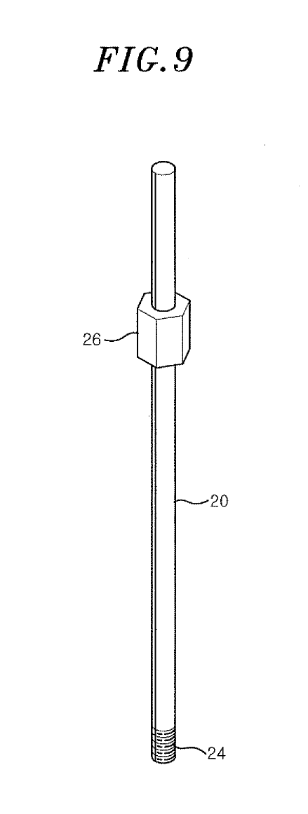

Referring to the drawings, the electrode system for cranial nerve stimulation in accordance with the first embodiment of the present invention includes a support coupling part 42, an electrode holder 40, and an anchor 60. The support coupling part 42 is formed to couple the support 20 (see, FIG. 9) thereto. The electrode holder 40 includes a stimulating electrode insertion part 44 formed to couple a stimulating electrode 6 to the vertical bottom of the support coupling part 42, and is coupled to a connecting wire 22 for supplying electricity to the stimulating electrode 6. The anchor 60 is fixed to the skull of a subject and includes an electrode holder insertion part 62 having a hollow part 64 into which the electrode holder 40 coupled to the connecting wire 22 is inserted.

The electrode holder 40 has a cylindrical shape and is formed of a non-conducting substance such as Teflon. The electrode holder insertion part 62 of the anchor 60 is formed in a cylindrical shape such that the hollow part 64 thereof corresponds to the shape of the electrode holder 40. The electrode holder 40 has a wedge-shaped guide groove 56 formed on the outer circumference thereof, and the electrode holder insertion part 62 of the anchor 60 has a wedge-shaped guide protrusion 72 formed in the hollow part 64 thereof and inserted into the wedge-shaped guide grooves 56 of the electrode holder 40. The guide groove 56 and the guide protrusion 72 guide the insertion of the electrode holder 40 into the anchor 60. Alternatively, the electrode holder 40 may have a guide protrusion formed on the outer circumference thereof, and the electrode holder insertion part 62 may have a guide groove formed in the hollow part 64 thereof.

In the electrode system in accordance with the first embodiment of the present invention, the electrode holder 40 and the hollow part 64 of the electrode holder insertion part 62 are formed in shapes corresponding to each other, and therefore the electrode holder 40 is stably coupled to the electrode holder insertion part 62. Further, the electrode holder 40 inside the electrode holder insertion part is prevented from being rotated by the guide members including the guide groove 56 and the guide protrusion 72. Although not explicitly illustrated in the drawings, alternatively, the electrode holder 40 may be formed to have an elliptical or polygonal cross-section, instead of the circular cross-section. In this case, the shape of the hollow part 54 of the electrode holder insertion part 62 may be formed to correspond to the shape of the electrode holder 40, and the guide groove 56 and the guide protrusion 72 may not be provided. In accordance with the first embodiment, the shape of the hollow part 62 of the electrode holder insertion part 62 in the anchor 60 may be changed according to the shape of the electrode holder 40.

As shown in FIGS. 4 and 5, the electrode holder 40 has a stimulating electrode insertion groove 46 which is formed in such a manner that the stimulating electrode 6 is disposed in the center of the stimulating electrode insertion part 44, and the stimulating electrode 6 is inserted into the stimulating electrode insertion groove 46. The stimulating electrode 6 is fixed by filling a solder housing part 48 formed under the stimulating electrode insertion groove 46 with solder in a state where the stimulating electrode 6 is electrically contacted with the connecting wire 22. Meanwhile, the connecting wire 22 is coupled to the solder housing part 48 through an opening part 50 for receiving the connecting wire formed in the side of the electrode holder 40.

The electrode holder insertion part 62 of the anchor 60 includes a connecting wire exit 66 for drawing out the connecting wire 22 coupled to the electrode holder 40 and a flange part 76 with a screw attachment hole 78.

Meanwhile, the electrode holder 40 has a pair of separation grooves 52 formed in both sides of the electrode holder 40 so as to face each other, and the electrode holder insertion part 62 of the anchor 60 has a separation groove exposure part 68 formed to expose the separation grooves 52. Therefore, when the electrode holder 40 is inserted into the hollow part 64 of the electrode holder insertion part 62, the separation grooves 52 of the electrode holder 40 are aligned with the separation groove exposure part 68. The electrode holder 40 may be separated from the anchor 60 using a tool such as a driver. One side of the separation groove exposure part 68 communicates with the connecting wire exit 66 connected to the connecting wire 22.

The electrode holder 40 has a plurality of fixing grooves, for example, four fixing grooves 54 formed on the upper outer circumference thereof. The electrode holder insertion part 62 has four fixing protrusions 74 formed to correspond to the fixing grooves 54. When the electrode holder 40 is inserted into the anchor 60, the fixing protrusions 74 are coupled to the fixing grooves 54 such that the electrode holder 40 and the anchor 60 are coupled to each other. Alternatively, the fixing grooves 54 and the fixing protrusions 74 serving as fixing members may be formed in the electrode holder insertion part 62 and the electrode holder 40, respectively.

Meanwhile, the support coupling part 42 of the electrode holder 40 has a screw part 58 formed to be coupled to the screw part 24 of the support 20, as illustrated in FIG. 9. The support 20 has a polygonal screw member 26 embossed thereon to facilitate the screw coupling and separation. However, it is understood that the polygonal screw member 26 may be engraved in the support 20.

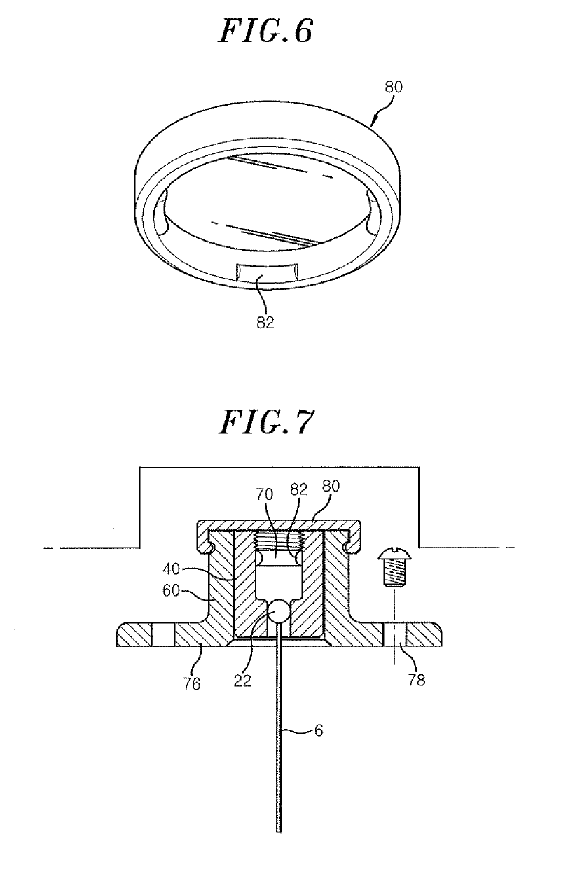

Referring to FIGS. 6 and 7, an assembly of the electrode holder 40 and the anchor 60 is covered by a cover 80. The cover 80 has attachment protrusions 82 formed inside thereof, and the anchor 60 has an attachment groove 70 (see, FIG. 3) formed on the outer circumference of the electrode holder insertion part 64 such that the attachment protrusions 82 are attached to the attachment groove. Alternatively, the attachment groove may be formed in the cover 80, and the attachment protrusions may be formed on the electrode holder insertion part 62.

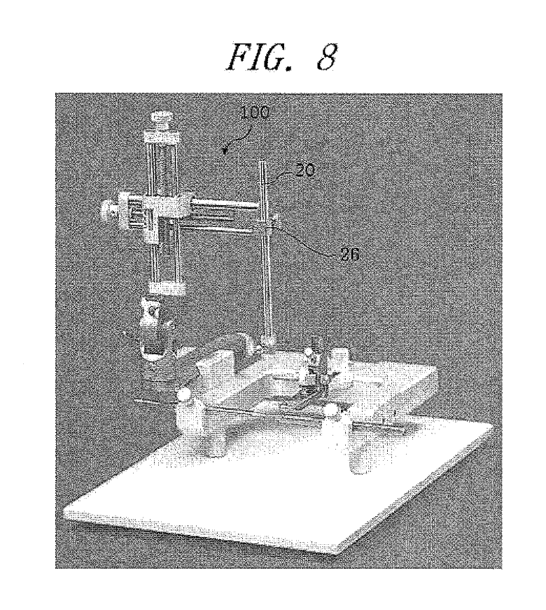

The electrode system having the above-described structure in accordance with the first embodiment of the present invention is transplanted into the skull of a subject using a three-dimensional stereotaxic instrument 100 as illustrated in FIGS. 8 and 9.

First, the stimulating electrode 6 is inserted into the stimulating electrode insertion groove 46 of the electrode holder 40, electrically contacted with the connecting wire 22, and fixed by filling the solder housing part 48 of the electrode holder 40 with solder. Then, the electrode holder 40 where the stimulating electrode 6 and the connecting wire 22 are electrically contacted with each other is inserted into the hollow part 62 of the anchor 60 so as to be coupled to the anchor 60. The screw part 24 of the support 20 is then coupled to the screw part 58 of the electrode holder 40.

Subsequently, as shown in FIGS. 8 and 9, the support 20 is attached to the stereotaxic instrument 100, and a target portion is then searched for. As well known in the art, stereotaxy refers to matching an image space with a physical space of reality and making a map of a necessary portion of the brain. Through the stereotaxy, a desired anatomic target or focus may be accurately accessed during transplantation, using three-dimensional spatial coordinates for the anatomic target or focus. The detailed descriptions of the operation of searching for the target portion using the stereotaxic instrument 100 is well known in the art and therefore will be omitted from this embodiment of the present invention. The stimulating electrode 6 is stuck in the target portion, and then fixed by coupling a screw to the screw coupling hole 78 formed in the flange part 76 of the anchor 60. Thereafter, the screw member 26 is rotated to separate the electrode holder 40 from the support 20, and the assembly of the electrode holder 40 and the anchor 60 is covered by the cover 80 and buried in the skull of the subject, and the incised portion is then sutured. Therefore, although the subject freely moves, the experiment may be reliably performed because the stimulating electrode is stably fixed during the experiment.

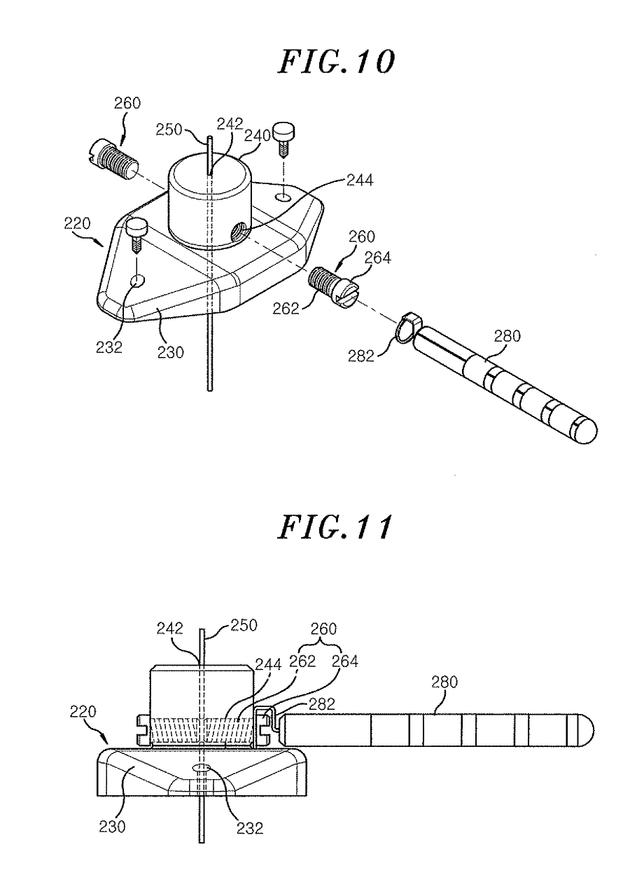

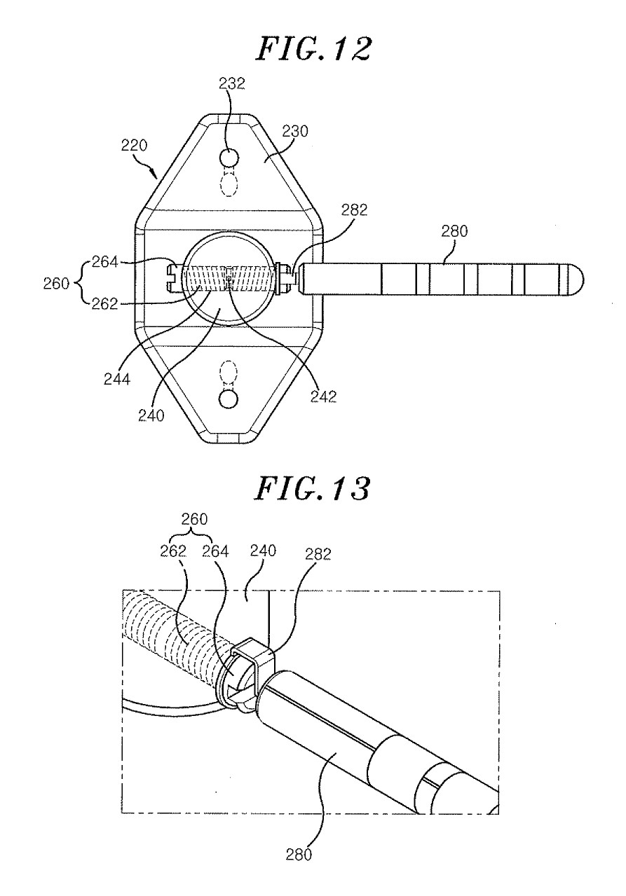

FIG. 10 is an exploded perspective view of an electrode system for cranial nerve stimulation in accordance with a second embodiment of the present invention, FIG. 11 is an assembled cross-sectional view of FIG. 10, and FIG. 12 is an exploded cross-sectional view of FIG. 10.

Referring to the drawings, the electrode system for cranial nerve stimulation in accordance with the second embodiment of the present invention includes an anchor member 220 fixed to the skull of a subject. The anchor member 220 has a cylindrical electrode holder 240 which supports a stimulating electrode 250 inserted therein, and a flange part 230 which is extended to both sides of the electrode holder 240 and formed integrally with the electrode holder 240.

The flange part 230 has screw holes 232 formed to fix the anchor member 220 to the skull of the subject. In this connection, the screw holes 232 may be inclined toward the electrode holder 240 so as to reliably fix the anchor member 220.

The electrode holder 240 may be formed of a non-conducting substance such as Teflon, and has an insertion hole 242 formed in the center thereof, into which the stimulating electrode 250 is vertically inserted, and two screw holes 244 formed to cross the stimulating electrode 250 at both sides of the body of the cylindrical electrode holder.

Stimulating electrode support screws 260 are coupled to the screw holes 244 at both sides of the electrode holder 240, and contacted with the stimulating electrode 250 inserted into the insertion hole 242 so as to support the stimulating electrode 250. Although it has been illustrated in the drawing that the pair of support screws 260 are coupled to the body of the cylindrical electrode holder so as to face each other and support the stimulating electrode 250, only one support screw 260 may be used to support the inserted stimulating electrode 250.

Each of the stimulating electrode support screws 260 includes a screw part 262 coupled to the screw hole 244 formed in the electrode holder 240 and a head part 264 protruding from the electrode holder 240. As illustrated in FIG. 13, a coupling part 282 of a connecting wire 280 is coupled to the head part 264 so as to supply current to the stimulating electrode 250.

As described above, the anchor member 220 includes the electrode holder 240 and the flange part 230 which are integrally formed, and is configured to couple the stimulating electrode support screw 260 connected to the connecting wire 280 to the screw hole 244 of the electrode holder 240, thereby fixing the stimulating electrode 250. Therefore, the electrode system in accordance with the second embodiment of the present invention has a simple structure in general and is convenient to use.

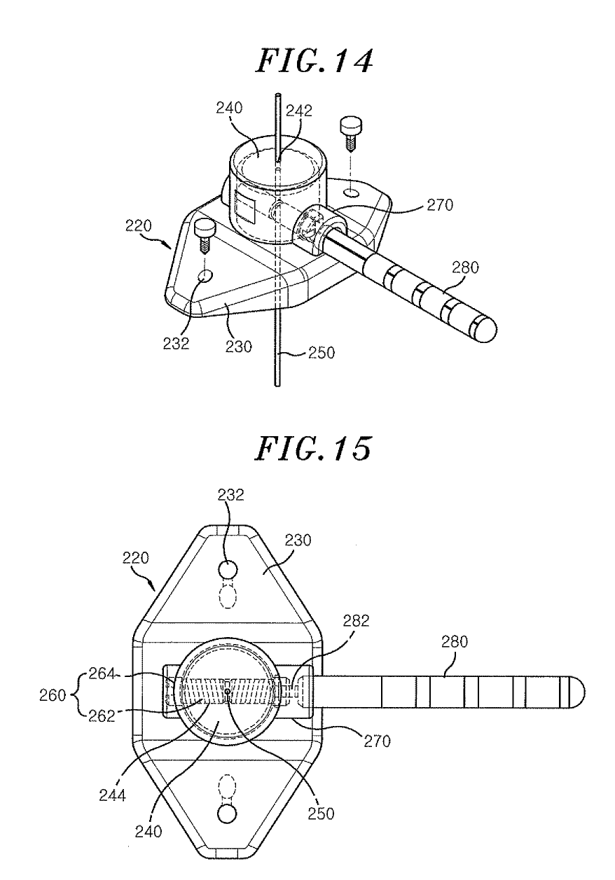

FIG. 14 is a perspective view of an electrode system for cranial nerve stimulation in accordance with a third embodiment of the present invention, and FIG. 15 is an assembled plan view of FIG. 14.

Referring to the drawings, the electrode system for cranial nerve stimulation in accordance with the third embodiment of the present invention has the same configuration as the electrode system for cranial nerve stimulation of the second embodiment illustrated in FIGS. 10 to 13, except that a cover 270 for covering the head part 264 of the stimulating electrode support screw 260 is integrally formed with the electrode holder 240 of the anchor member 220, in order to prevent the head part 264 from being exposed when the stimulating electrode support screw 260 is coupled to the electrode holder 240 such that the connecting wire 280 is coupled to the head part 264 protruding from the electrode holder 240. Hence, the detailed descriptions thereof will not be made herein.

The electrode system having the above-described structure of the embodiment is transplanted into the skull of a subject using the three-dimensional stereotaxic instrument 100 illustrated in FIGS. 8 and 9. In the first embodiment of the present invention, it has been described that the screw part 58 of the electrode holder 40 is coupled to the screw part 24 of the support 20 illustrated in FIG. 9 so as to mount the electrode holder 40 on the stereotaxic instrument 100. However, in the electrode system in accordance with the second and third embodiments of the present invention, a structure (not illustrated) for holding the stimulating electrode 250 of the electrode holder 240 is provided instead of the screw part 24 of the support 20, in order to mount the electrode holder 240 on the stereotaxic instrument 100.

First, the stimulating electrode 250 is inserted into the insertion hole 242 of the electrode holder 240, and a target portion is searched for by the stereotaxic instrument 100. Then, the stimulating electrode 250 is stuck in the target portion, and the anchor member 220 is fixed by coupling screws to the screw holes 232 formed in the flange part 276 of the anchor member 220. Thereafter, the stimulating electrode support screw 260 having the head part 264 coupled to the coupling part 282 of the connecting wire 280 is coupled to the screw hole 244 of the electrode holder 240 and electrically contacted with the stimulating electrode 250. The electrode holder 240 and the head part 264 of the stimulating electrode support screw 260 are then covered by the cover 270 to insulate the structure. The assembly is then buried in the skull of the subject, and the incised portion is sutured. Therefore, although the subject freely moves, the experiment may be reliably performed because the stimulating electrode is stably fixed during the experiment.

While the invention has been shown and described with respect to the embodiments of the electrode system for cranial nerve stimulation, the present invention is not limited thereto. It will be understood by those skilled in the art that various changes and modifications may be made without departing from the scope of the invention as defined in the following claims.

1. An electrode system for cranial nerve stimulation, comprising:
a support coupling part for coupling a support, wherein the support coupling part having a stimulating electrode insertion part formed to couple a stimulating electrode at the bottom thereof;
an electrode holder formed to couple to a connecting wire for supplying electricity to the stimulating electrode; and
an anchor having an electrode holder insertion part formed with a hollow part into which the electrode holder coupled to the connecting wire is inserted, and fixed to the skull of a subject.

2. The electrode system of claim 1, wherein the electrode holder comprises:
a stimulating electrode insertion groove formed in the center of the stimulating electrode insertion part such that the stimulating electrode is inserted into the stimulating electrode insertion groove;
a solder housing part formed under the stimulating electrode insertion groove so as to be electrically contacted with the connecting wire; and
an opening part for receiving the connecting wire formed in the side of the electrode holder so as to couple the connecting wire to the solder housing part.

3. The electrode system of claim 1, wherein the electrode holder insertion part of the anchor comprises a connecting wire exit for drawing out the connecting wire coupled to the electrode holder, and a flange part having a screw coupling hole.

4. The electrode system of claim 1, wherein the electrode holder comprises a pair of separation grooves formed in both sides thereof so as to face each other, and a separation groove exposure part formed to expose the separation grooves when the electrode holder is inserted into the hollow part of the anchor.

5. The electrode system of claim 4, wherein one side of the separation groove exposure part is configured to communicate with the connecting wire exit connected to the connecting wire.

6. The electrode system of claim 1, wherein the electrode holder and the anchor include fixing members provided to respectively correspond to each other, by which the electrode holder is inserted into the anchor and coupled to the anchor.

7. The electrode system of claim 1, further comprising a cover for covering an assembly of the electrode holder and the anchor,
wherein the cover and the assembly are coupled to each other by attachment members which are formed in the cover and the anchor, respectively, so as to correspond to each other.

8. The electrode system of claim 1, wherein the electrode holder is inserted into the anchor by guide members which are respectively formed in the electrode holder and the anchor, respectively, so as to correspond to each other.

9. The electrode system of claim 1, wherein the support coupling part of the electrode holder is screwed to the support.

10. An electrode system for cranial nerve stimulation, comprising:
an anchor member configured to be fixed to the skull of a subject, wherein the anchor member includes a flange part having screw holes formed therein and an electrode holder formed integrally with the flange part such that a stimulating electrode is inserted into the electrode holder and supported by the electrode holder; and
a stimulating electrode support screw coupled to the electrode holder of the anchor member so as to support the inserted stimulating electrode, and coupled to a connecting wire for supplying electricity.

11. The electrode system of claim 10, wherein the stimulating electrode support screw comprises a screw part and a head part, the screw part is coupled to a screw hole formed in the electrode holder, and the head part is coupled to the connecting wire.

12. The electrode system of claim 10, wherein the stimulating electrode support screw comprises a pair of stimulating electrode support screws, each electrode support screw having a screw part and a head part, and wherein each of the screw parts is coupled to a screw hole formed in the electrode holder, and one of the head parts is coupled to the connecting wire.

13. The electrode system of claim 12, further comprising a cover formed integrally with the electrode holder of the anchor member so as to cover the head part of the stimulating electrode support screw coupled to the connecting wire.
